(19) United States
(12) Patent Application Publication
(10) Pub. No.: US 2024/0350055 A1
(43) Pub. Date: Oct. 24, 2024

(54) METHOD AND SYSTEM FOR CALIBRATING A HUMAN STATE SENSOR

(71) Applicant: Harman International Industries, Incorporated, Stamford, CT (US)

(72) Inventors: Andrey Viktorovich FILIMONOV, Kamenki (RU); Ivan Sergeevich SHISHALOV, Nizhniy Novgorod (RU); Anastasiya Sergeevna FILATOVA, Balakhna (RU); Anzhela Grigorevna BUROVA, Nizhny Novgorod (RU); Evgeny Pavlovich BURASHNIKOV, Nizhny Novgorod (RU); Oleg Andreevich GROMAZIN, Nizhniy Novgorod (RU); Anastasiya Vladimirovna BAKHCHINA, Nizhniy Novgorod (RU); Mikhail Sergeevich KLESHNIN, Bol`shoe Mokroe (RU)

(21) Appl. No.: 18/759,157

(22) Filed: Jun. 28, 2024

Related U.S. Application Data
(63) Continuation-in-part of application No. PCT/RU2021/000604, filed on Dec. 28, 2021.

(51) Int. Cl. A61B 5/18; B60W 30/16; B60W 50/14; G06F 17/18

(52) U.S. Cl. CPC A61B 5/18; B60W 30/16; B60W 50/14; G06F 17/18; A61B 2560/0223; B60W 2050/143; B60W 2050/146

(57) ABSTRACT

There is provided a method and system for calibrating a human state sensor, the method comprising: detecting a plurality of output values of the sensor, the output values being indicative of a mental and/or physiological state of a user; determining a boundary output value based on the detected plurality of output values, wherein a predetermined part of the detected output values is higher or lower than the boundary output value; determining a threshold output value of the sensor based on the boundary output value.

### CROSS REFERENCE TO RELATED APPLICATIONS

The present application is a continuation-in-part U.S. National Phase of International Application No. PCT/RU2021/000604, entitled “METHOD AND SYSTEM FOR CALIBRATING A HUMAN STATE SENSOR”, and filed on Dec. 28, 2021. The entire contents of the above-listed application are hereby incorporated by reference for all purposes.

### FIELD

The present disclosure relates to human state sensors, such as mental and/or physiological state sensors, and the calibration thereof, in particular to personalizing human state sensors.

### BACKGROUND

Human mental, physiological or physiological state sensing is a rapidly developing area, in particular within the automotive domain. Better state sensing allows, for example, for enhancing driving safety and comfort of a user of a vehicle and may also leverage well-being and health applications. Moreover, it may assist to explore vehicles as yet another living space, especially regarding health diagnostics.

A human mental, physiological or psychological state is a highly personalized. Different human beings have different backgrounds and are used to manage problems with different efficiency. Environmental conditions influence different human beings differently; what makes some user stress is an easy task for others; what requires a significant cognitive effort for ones is a routine for others.

Hence, an improved and/or more reliable sensor or sensing method is desired that takes into consideration personalized mental, physiological or psychological state indicators or symptoms.

### SUMMARY

According to one of many embodiments, there is provided a method for calibrating a mental and/or physiological state sensor, the method comprising: detecting a plurality of output values of the sensor, the output values being indicative of a mental and/or physiological state of a user; determining a boundary output value based on the detected plurality of output values, wherein a predetermined part of the detected output values is higher or lower than the boundary output value; determining a threshold output value of the sensor based on the boundary output value.

For example, the personalized threshold output value may be determined as the boundary output value or may be determined as the boundary output value plus a (predetermined and/or dynamic) offset value or offset value range. In other words: A guard band around the boundary value may be provided. The threshold output value may thus represent a personalized threshold output value. The threshold output value may be (usable) for determining a threshold (e.g. a critical) mental and/or physiological state of the user. In other words: The method may further comprise: Determining a mental and/or physiological state of the user based on the determined threshold output value. Thereby, the method is extended to a method for determining a human state of a user via a mental and/or physiological state sensor.

The invention is based on the finding that the human state, e.g. the mental state, which may also be referred to as a psychological state, and/or the physiological state, which may also be referred to as a (physical) health state, may surface differently for different human beings. Beyond that, every human being may react differently on different situations. By taking into account previously detected sensor data or historical sensor data of a certain human being or user, the human state (mental and/or physiological state) sensor can be specifically calibrated, i.e. personalized for said certain user. Beyond that, the defined calibration method may be continuously performed, even during use of the sensor, thereby being continuously controlled and improved. In that way, the validity of the sensor is enhanced.

The sensor may be located within a vehicle. In other words, the user may be driver of the vehicle.

According to an embodiment, the method further comprises: determining a frequency of detection for each of the detected plurality of output values; and determining the boundary threshold output value based on the determined frequencies of detection.

The frequency of detection may represent a frequency of detection function, such as a value density function or a value histogram. By way of considering limits or border areas of historical sensor data via the boundary output value, wherein a predetermined part or percentage, such as 1% or 5%, of all detected sensor output values is higher (or lower) as said boundary output value, the calibration method takes into account personal fluctuations of detectable human state indicators or symptoms. Hence, a simple and robust way of calibrating or personalizing the sensor is provided, thereby further enhancing the validity of the sensor.

According to an embodiment, the method further comprises: determining, based on the determined frequencies of detection, an integral distribution of the detected output values over an output value range; and determining the boundary output value based on the determined integral distribution.

The output value range may be a detected, estimated, expected or predetermined output value range. The integral distribution may be an integral (function) of the frequency of detection (function) for the detected output values. In other words: The integral distribution may be an integral density function or an integral value histogram. The integral distribution function may indicate, for a certain detected output data value, the percentage or part of determined output data values that are greater or smaller than the certain determined output data value. Thereby, an effective, simple and reliable way of determining the boundary output is provided.

According to an embodiment, the integral distribution is an integral distribution function defined by

$$IH ( i ) = \frac{{\sum }_{j ∈ \{ j \vert j \geq i \}} H ( j )}{N} ,$$

wherein IH(i) is an integral distribution function value for a detected output value i, H(j) is the determined frequency of detection for a detected output data value j, and N is a total number of detected output values. Alternatively, the integral distribution function may be a continuous integral distribution function defined by

$$IH ( i ) = \frac{{\int }_{i}^{max} H ( j ) dj}{{\int }_{min}^{max} H ( j ) dj} ,$$

wherein IH(i) in both discrete and continuous forms indicates the frequency of detection for output values greater than i. Similarly, the discrete and continuous integral distribution function values IH(i) may indicate the frequency of detection for output values smaller than i. Thereby, an effective, simple and reliable way of determining the integral distribution is provided.

According to an embodiment, the output values are output value ranges of output values, in particular output value ranges of a predetermined width

The plurality of value ranges may be within and/or extend across a (total) output value range of the sensor. In other words, each output value may be assigned to a corresponding value range. In that manner, a subgroup of output values may be considered as one output value or one group of output values. Thereby, the processing power or time needed to perform the method is reduced. In other words, the efficiency of the method is increased.

According to an embodiment, the plurality of value ranges have different widths, in particular wherein the plurality of value ranges are decreasing in size for lower or higher output values.

For example, the plurality of value ranges are decreasing in size the closer they are to the boundary value or an estimated boundary value. In that manner, a better resolution in a higher (or lower) output value area, in particular in an area relatively close to, e.g. next to, the boundary output value, can be achieved.

According to an embodiment, the method further comprises: determining the number of value ranges based on a predetermined accuracy requirement of the threshold value, in particular based on the predetermined part of the detected output values that are higher or lower than the boundary output value.

For example, if the total number of value ranges is 10, e.g. the total range of detected (or detectable) output values is divided by 10 and the topmost (i.e. highest or lowest) value range comprises 4% of the detected value ranges although a boundary output value is required that is higher (or smaller) than 99% of the detected output values, the number of value ranges is to be increased. Thereby, the processing power or time needed to perform the method is reduced whilst taking into account accuracy requirements.

According to an embodiment, the method further comprises: determining whether the detected plurality of output values meets a first threshold number of output values, in particular within a total output value range of the sensor and/or within one or more output value ranges, more particularly above or below the determined boundary output value; and determining the threshold output value based on the boundary output value if the first threshold number is met.

According to an embodiment, the method further comprises: detecting the plurality of output values of the sensor within a predetermined time interval; and determining the threshold output value based on the boundary output value after the predetermined time interval has expired.

In that manner, it can be ensured that the detected historical data provides a significant basis for determining the personalized threshold output value.

According to an embodiment, the method further comprises: determining whether a second threshold number of output values has been detected in one or more specific, in particular different, sensor environments, user scenarios and/or time frames; and determining the threshold output value based on the boundary output value if the second threshold number is met.

In that manner, it is ensured that the detected historical data is detected with respect to typical situations and/or a user's experience the sensor or sensor system will then operate in. For example, the environments, user scenarios and/or time frames comprises one or more regular commutes of the user (e.g. between a workplace and a home), typical routes (e.g. types of routes such as highways and urban areas), typical times of day, and/or cognitive load scenarios, such as a user making a call, being cognitive distracted or daydreaming (e.g. heavy inner cognition induced by thinking over a problem) during driving. Hence, a meaningful, i.e. realistic basis for determining the threshold output value is provided.

According to an embodiment, the method further comprises: defining the threshold output value based on a predetermined boundary output value if the first and/or second threshold values are not met and/or if the time interval has not expired.

It is thereby ensured that the calibration method allows for an interpretation of the sensor output values even if not enough historical data has yet been detected.

According to another embodiment, there is provided a device comprising a processor configured to perform the above described method. The device may be arranged in or comprised by a vehicle.

According to another embodiment, there is provided a computer program comprising instructions which, when the program is executed by a computer, cause the computer to carry out the above described method. The computer may be arranged in or comprised by a vehicle.

According to another embodiment, there is provided a non-transitory computer-readable storage medium comprising instructions which, when executed by a computer, cause the computer to carry out the above described method.

### BRIEF DESCRIPTION OF THE DRAWINGS

The features, objects, and advantages of the present disclosure will become more apparent from the detailed description of non-limiting embodiments set forth below when taken in conjunction with the drawings in which like reference numerals refer to similar elements, wherein:

FIG. 1 shows a flowchart of a method for calibrating a mental and/or physiological state sensor,

FIG. 2 shows a flowchart of a method for improving validity of the calibration method,

FIG. 3 shows a data-processing device including the sensor, a memory and a processor,

FIG. 4 shows exemplary output values of the sensor,

FIG. 5 shows a frequency of detection histogram for an exemplary output value,

FIG. 6 shows a first frequency of detection histogram for all exemplary output values within an exemplary total output value range,

FIG. 7 shows a first exemplary integral distribution histogram of the first frequency of detection histogram,

FIG. 8A shows a second frequency of detection histogram with output value ranges of different widths for all exemplary output values within the exemplary total output value range,

FIG. 8B shows a second exemplary integral distribution histogram of the second frequency of detection histogram with output value ranges of different widths,

FIG. 9 shows the exemplary output values of the sensor with determined 5% of critical output values,

FIG. 10 shows a third exemplary integral distribution histogram of the second frequency of detection histogram, and

FIG. 11 shows a flowchart of a method for determining human states.

### DETAILED DESCRIPTION OF THE PREFERRED EMBODIMENTS

The following description relates to systems and methods for calibrating human state detection to an individual. Human mental, physiological or physiological state sensing is a rapidly developing area, in particular within the automotive domain. Human state sensing allows, for example, for enhancing driving safety and comfort of a user of a vehicle and may also leverage well-being and health applications. However, assessing a user's (e.g., a driver's) mental state based on predefined parameters may not be individualized and therefore erroneous detections may occur.

As an example, heart rate may be a parameter that is detected during determination of human state. Predefined parameters may indicate that a heart rate of 45 bpm indicates a drowsy state, a heart rate of 60 bpm may indicate a baseline alert state, a heart rate of 90 bpm may indicate a strained state, and a heart rate of 120 bpm may indicate a stressed state (e.g., a high anxiety state). However, these predefined parameters and thresholds may not be applicable to all users. For example, a user may have a baseline heart rate of 90 bpm and a drowsy heart rate of 60 bpm, in which case, comparison of such values may indicate that the user is alert at baseline when they are actually falling asleep while driving.

Further, different users may have different responses to particular scenarios. For example, a first user's heart rate may spike in a given scenario but a second user's heart rate may remain stagnant in the same given scenario. Thus, action taken to avoid the scenario when that scenario is detected may be recommended for the first user but may not be necessary for the second user as the triggering threshold for the second user may be higher than the first user.

Thus, a method for calibrating and personalizing human state detection is herein disclosed. The method as herein provided uses both user and sensor behaviour by gathering historical data of the sensor outputs and then calculating personalized thresholds for the user. By gathering historical records with sensor outputs, building a set of the values (e.g., as a histogram), and processing the set of values (e.g., building an integral value histogram), a threshold may be defined for the user. The threshold range may then be used when determining human states of that particular driver. The method herein presented may be performed on an individual basis, for example for each vehicle or for each driver profile of a vehicle. Thus, human state detection may be more accurate for each individual driver. Further, the method may be performed in an iterative manner such that the threshold range is repeatedly calibrated for changing detectable parameters of the user.

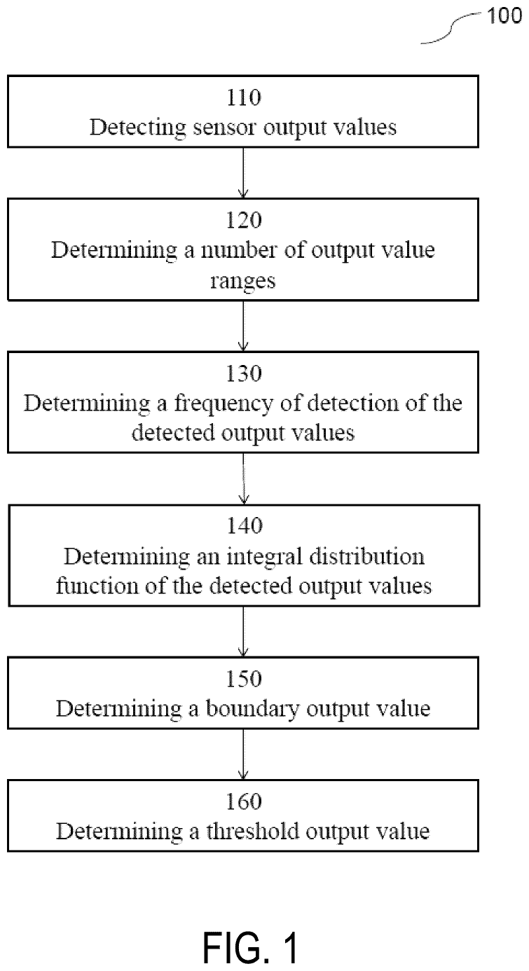

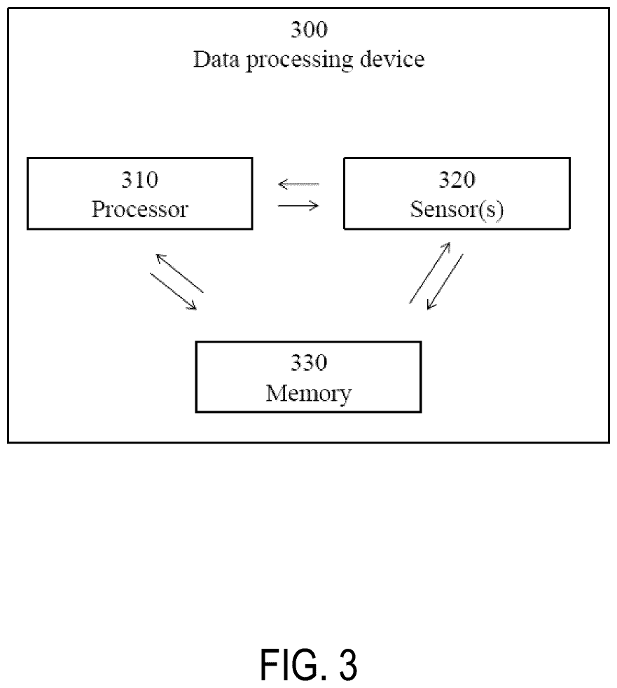

Starting with FIG. 1, a flowchart illustrating a method 100 for calibrating a mental and/or physiological state sensor, also referred to as a human state sensor, is shown. The described method steps may be performed in any suitable different order. The method 100 may be executed by one or more processors according to instructions stored in non-transitory memory. For example, the method 100 may be executed by processors 310 according to instructions stored in memory 330 of FIG. 3, as will be described further below.

In step 110, a plurality of output values of the sensor are detected. The human state sensor or detector may have a continuous output within a time interval. The output values of the sensor may correspond to a human state of a user, i.e. of a mental and/or physiological state of the user. More particularly, the output values are indicative of an intensity of the state being detected depending on the amount of, or corresponding, to the output value. The output values extend over a certain output value range. In a preferred embodiment, the plurality of output values comprises more than two different output values. As an example, a heart rate or heart rate pattern of the user may be detected.

In some examples, the output values of the sensor may be historical record data and may thus represent sensor outputs for a variety of typical driving scenarios. Further, the historical record data may also include not only typical external conditions, but also typical internal ones. For example, the user may often take calls while driving, text while driving, speak to passengers while driving, and the like, that may affect their mental state.

In step 120, a number of output value ranges is determined within a total value range of the sensor or within a total value range of detected output values. In other words, the output values detected in step 110 are grouped into sub-ranges. Additionally or alternatively, detectable output values (i.e. an output value range of the sensor) is divided into sub-ranges. For example, a range of [0 . . . 1] is split into three sub-ranges [0 . . . 0.33], (0.33 . . . 0.66] and (0.66 . . . 1]. Put in yet another way, detected output values within a sub-range are represented by one of the values within the sub-range, for example the highest, the lowest or an average output value within the sub-range. The sub-ranges within the output value range may have equal widths or sizes. Alternatively, the sub-ranges may have different sizes, in particular may be smaller or larger for higher or lower sub-ranges. Determining the sub-ranges may be a first step of processing the historical recorded data.

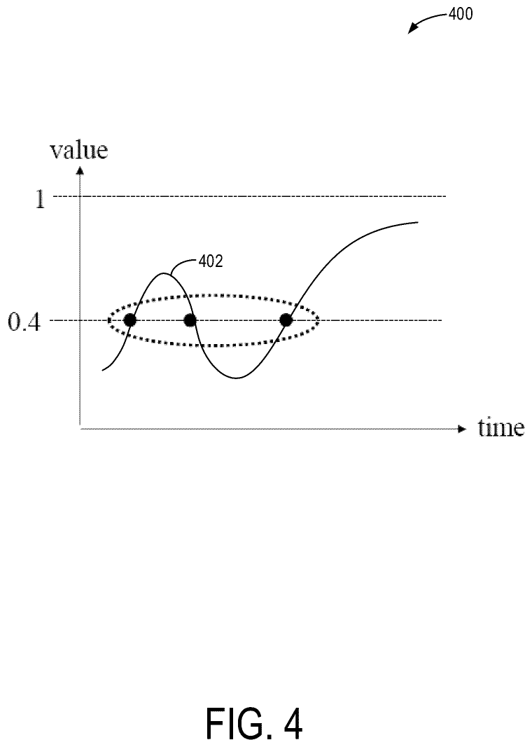

Briefly referring to FIG. 4, where exemplary output values of the sensor are presented over time in a graph 400. A plot 402 is shown within the graph 400, indicating output value of the sensor over time. It is illustrated that detector output values of 0.4 and approximately 0.4 are grouped together to a sub-range of output values of 0.4. In other words, values that are proximate to 0.4 are rounded to 0.4. In the present example, all output values that are detected three times within the shown time interval are each rounded to three separate values of 0.4. Put differently, output values that are proximate to 0.4 (including the output value at 0.4) that are detected at a time, directly after each other or consecutively, are considered as one output value 0.4.

Returning to FIG. 1, in step 130, a frequency of detection of the detected output values is determined. In other words, it is determined how often each output value as been detected within a (predetermined) time interval. Determining the frequency of detection of a particular value may give rise to a histogram for that value. The histograms for each individual value may then be used to generate a histogram for multiple values.

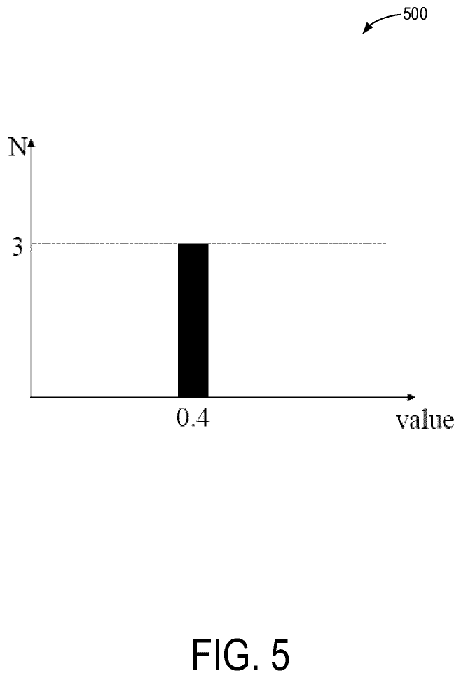

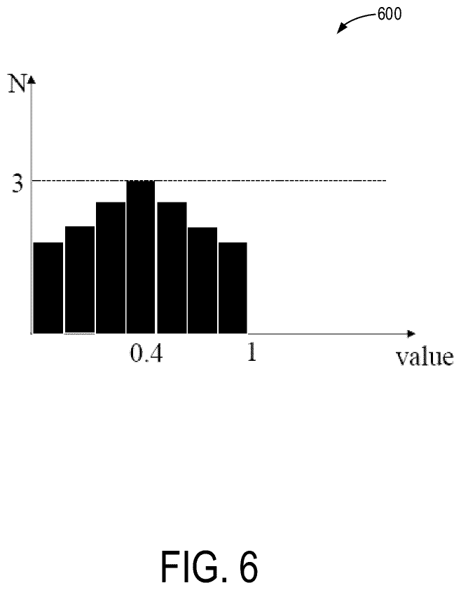

For example, as shown in FIG. 4, the plot 402 includes three values of 0.4, which may be its frequency. Turning briefly to FIG. 5, a frequency of detection histogram 500 (e.g., a frequency histogram) for the value 0.4 is shown. It is illustrated that the output value or sub-range 0.4 has been detected three times within the illustrated time interval. FIG. 6 shows a second frequency histogram 600 that plots frequencies of detection for multiple values. As illustrated in FIG. 6, the frequency of detection is determined for every sub-range within the range of detected output values. In the present example, seven equally distanced sub-ranges have been determined for that purpose. For example, the frequency of detection of the sub-range value 0.4 may be three while the frequency of detection of the sub-range value 0.8 may be two.

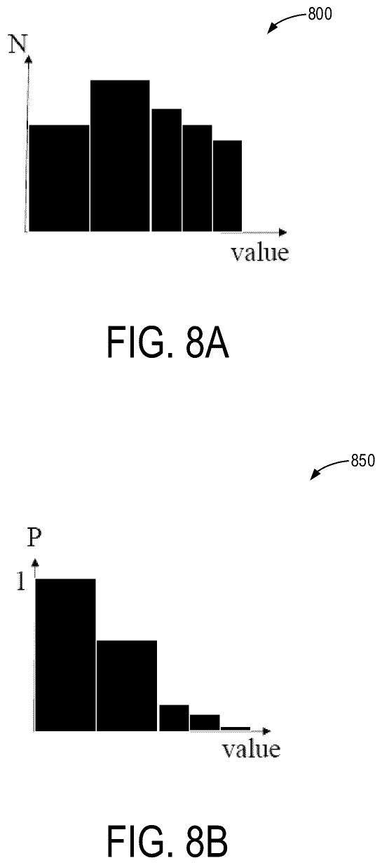

In some examples, as is shown in FIG. 6, each of the sub-ranges (e.g., bins) may have equal widths. In other examples, as illustrated in FIG. 8A, the sub-ranges may have different sizes. For example, if top values are aimed to be considered, the higher value sub-ranges, may be smaller to allow for better resolution in the top-value part of the histogram. For example, bins to the right of a frequency histogram 800 shown in FIG. 8A may be narrower (e.g., have a smaller width) than bins to the left.

Returning again to FIG. 1, in step 140, an integral distribution function of the detected output values is determined. The integral distribution function may be an integral value histogram.

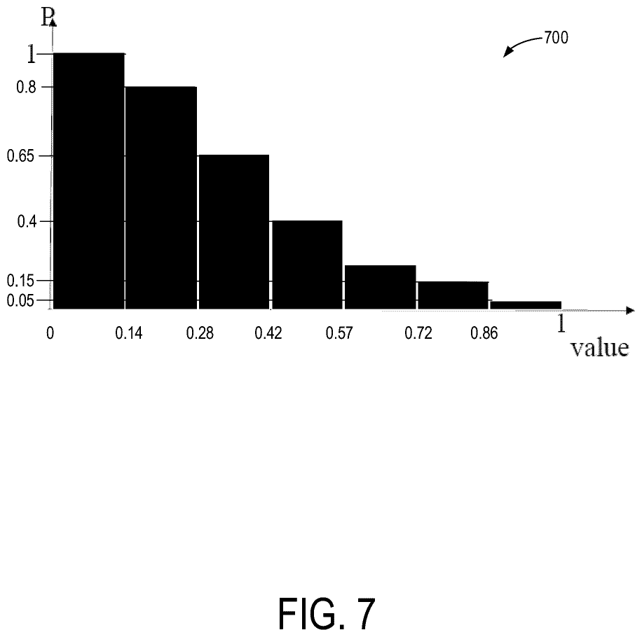

Referring briefly to FIG. 7, an exemplary integral value histogram 700 is shown. The integral value histogram 700 may be determined based on the previously determined frequency of detection of sensor output values, as illustrated in FIG. 6. FIG. 8B shows an exemplary histogram 850 that is determined based on the previously determined frequency of detection of sensor output values for unequal bins, as illustrated in FIG. 8A.

As shown, the integral distribution function can be described as indicating, for a certain output value or sub-range of output values, the number, part or percentage of other output values that lie below or above said certain output value. For example, the illustrated histogram shows values distributed between 0 and 1. Considering the leftmost sub-range, 100% of output values are greater than output value 0. Considering the rightmost sub-range, 0% of output values are greater than output value 1. Put in yet another way, the integral distribution indicates, for a certain output value or sub-range of output values, the sum over all frequencies of detection for output values above or below (and including) the certain output value or sub-range of output values, preferably divided by the total number of detected output values. As a formula, such an integral distribution may be expressed as shown in equation (1):

$$IH ( i ) = \frac{{\sum }_{j = i}^{M} H ( j )}{N} \qquad ( 1 )$$

wherein IH(i) is an integral distribution function value for a detected output value or sub-range i, N is a total number of detected output values, and M is a number of bins (e.g., a number of sub-ranges).

Each bin of the resulting integral value histogram may reflect how many (e.g., what part or percentage) of the output values have values greater than its left bound. For example, referring again to FIG. 7, the value distribution may be between 0 and 1. The leftmost bin may represent values between 0 and 0.14, with a left bound of 0. Thus, as is shown, 100% of the output values are greater than 0. However, the rightmost bin may represent values between 0.86 and 1. As is shown, 5% or less of the output values are greater than its left bound of 0.86.

Referring again to FIG. 1, in step 150, a boundary output values is determined. The boundary output value may be determined based on the determined integral distribution, wherein a predetermined part of the detected output values is higher or lower than the boundary output value. For example, the boundary output value may be determined such that 5% of the detected output values is higher than the boundary output value. More particularly, a certain value of the integral distribution that is associated with a percentage of 5% or less than 5% of detected output values that are higher or lower than said certain value may be chosen as the boundary output value.

In the case of the histogram presented in FIG. 7, the right most bin of values between 0.86 and 1, the associated percentage of values higher may be 5%, and thus a left bound value of the right most bin may be the boundary output value.

If none of the sub-ranges of the integral distribution is associated with a predetermined or desired part or percentage of the detected output values that lie higher or lower than said sub-ranges, the method may return to step 120, where a different, in particular higher, number of output value ranges is determined. In that manner, the number of value ranges may be based on an accuracy requirement of the threshold value.

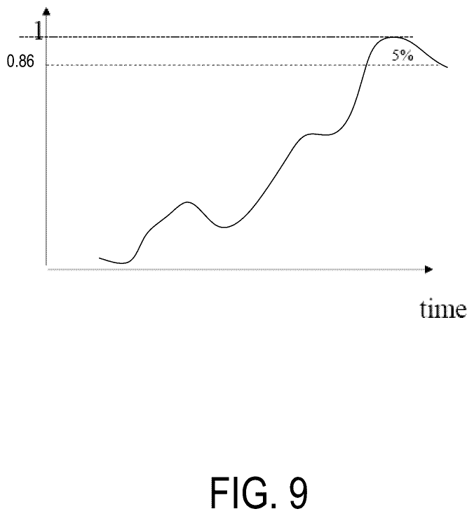

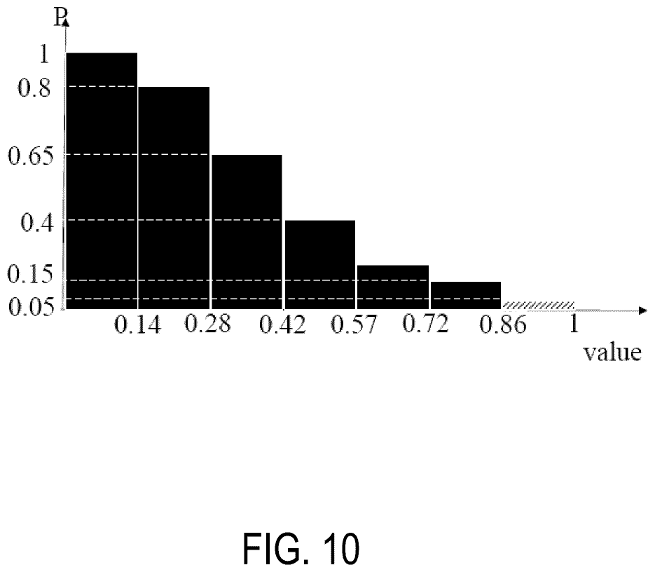

Referring to FIGS. 9 and 10, it is shown that in the present example the boundary output value may be set to 0.86. The integral histogram shown in FIG. 10 indicates that the sub-range between 0.86 and 1, e.g. [0.86, 1) or [0.86, 1] or (0.86, 1] or (0.86, 1), includes 5% of all detected output values that are greater than 0.86. In other words, 95% of all detected output values lie below the boundary output value 0.86.

Referring again to FIG. 1, in step 160, a threshold output value for the sensor is determined based on the previously determined boundary output value. The threshold output value may be set to, i.e. be equal to, the boundary output value (e.g., of 0.86, in the example presented in FIG. 7). Alternatively, the threshold output value may comprise an offset to the boundary output value, wherein the offset may be predetermined and fixed for every boundary output value or may depend on the (absolute) boundary output value or its amount.

Based on the threshold output value, a critical output value that lies above the threshold output value can be detected. In that manner, a critical mental or physiological state of the user may be detected. Hence, normal or uncritical fluctuations of sensor output values may be ignored, wherein only outliers of output values may be determined to be critical. In that manner, both user and sensor behaviour or properties are considered when interpreting sensor output values. In this way, it is possible to also use driver behaviour or properties as a way to reduce the amount of sensor output values taken into account for adjusting vehicle operation, thereby providing more efficient processing of the sensor output values for controlling vehicle operation since less sensor output value data is actually processed for controlling vehicle operation. This further can also enable a faster reaction by the vehicle when indeed the sensor output values become critical since the processor ignores non critical values.

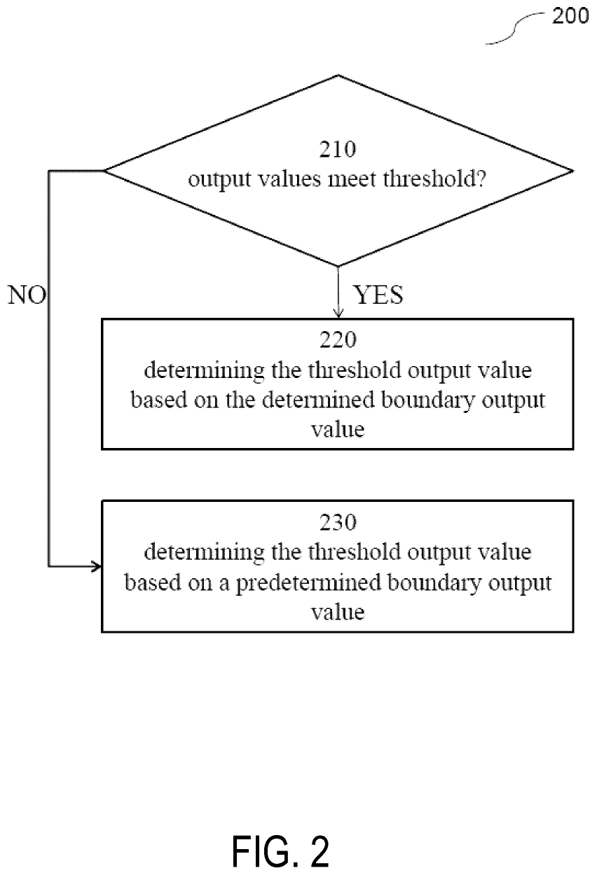

FIG. 2 continues the method 100 of FIG. 1. The further steps 200 may also be referred to as a method 200 for improving validity of the calibration method 100 of FIG. 1. The method 200 may be included in method 100 between any method steps 110 to 160 of method 100, in particular after step 110.

Method 100 starts with method step 210, where the method 200 determines whether the output values detected in step 110 of method 100 meet a threshold. The threshold may be a first threshold number of output values. The first threshold number of output values may be a total number of output values within the full range of detected or detectable output values or may be a total number of output values within a sub-range of detected or detectable output values, for example the highest or lowest sub-range or a sub-range higher or lower than a determined, expected or estimated boundary output value. In other words it is determined in step 210 whether enough output values have been detected in order to determine a valid or suitable threshold output value.

Alternatively or additionally, the threshold of step 210 may be a second threshold number of output values detected in different sensor environments, different user scenarios and/or at different timeframes or intervals during a (24 hours) day. For example, the second threshold number may indicate a minimum number of output values to be detected in a vehicle (i) during rush-hour (ii) during phone calls performed by the user (iii) during workdays or weekend, (iv) during a commute to or from a workplace of the user (v) on specific routes and/or (vi) during a specific night or day time. In other words, it is ensured that enough output values are detected during different mental and/or physiological states, in particular stress states, of the user in order to obtain a representative set of historical output values based on which the threshold output values determined.

Alternatively or additionally, the threshold of step 210 may be a time interval within which the plurality of output values are detected. In other words, it is determined whether output values of the sensor have been detected for a specific time period, in particular a time period that is long enough to allow for the detection of a significant number of output values.

If it is determined in step 210 that the threshold is met (yes branch), the threshold output value is determined based on the determined boundary output value as described above with respect to FIG. 1. If it is determined in step 210 that the threshold is not (yet) met (no branch), the threshold output value is determined based on a predetermined boundary output value. Alternatively, the boundary output value may be determined based on predetermined or pre-calculated reference data. Alternatively, the threshold output value may be set to a predetermined, expected or estimated threshold output value.

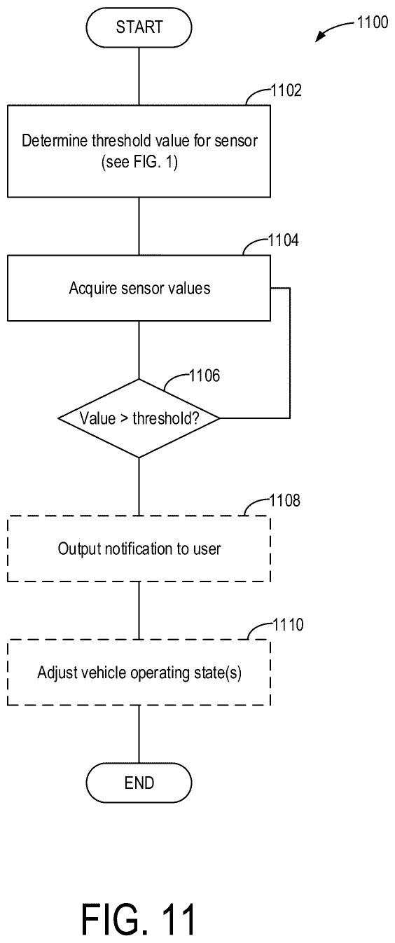

In this way, per method 100, a personalized threshold may be determined for the sensor. The threshold may indicate that a triggering event is occurring, in some examples, based on the human or mental state of the user. For example, if the output values of the sensor correspond to detected heart rate, with higher values corresponding to higher heart rates, the threshold value of the sensor may be tuned for the specific user based on their usual baselines during driving and typical responses to various scenarios. The threshold that is determined, as herein presented, may indicate a threshold for when an action may be taken, as will be described further with respect to FIG. 11. For example, when a threshold value for a sensor that detects mental state based on heart rate is met, a vehicle computing system may register the event and output a notification to the user. For example, a notification stating “high stress state detected, consider pulling over” may be outputted when the sensor outputs a value above the threshold. In another example, advanced driver assistance systems (ADASs) may be configured to adjust one or more operating states of the vehicle in response to output of a value above the threshold.

Turning briefly to FIG. 11, a flowchart illustrating a method 1100 for determining human states is shown. The method 1100 as herein presented may be executed by one or more processors according to instructions stored in non-transitory memory. For example, the method 100 may be executed by processors 310 according to instructions stored in memory 330 of FIG. 3, as will be described further below.

At 1102, method 1100 includes determining a threshold value for a sensor. The sensor may be a sensor, detector, evaluator, or the like that is configured to detect a particular parameter such as eye gaze, heart rate, or the like. The sensor output values may indicate human state (e.g., mental state, distraction state, etc.). For example, for a heart rate sensor, a higher sensor output value may correspond to a higher detected heart rate.

As described with respect to FIG. 1, historical record data of sensor output values may be obtained, a histogram of sensor output values may be generated, an integral value histogram may be generated based thereon (e.g., via equation (1)), and then based on the integral value histogram, a threshold value may be determined. The threshold value may be the value that 5% of the output values are greater than, as described above. The threshold value may thus indicate a level at which sensor output values may be considered relevant.

At 1104, method 1100 includes acquiring sensor values. The sensor values may be acquired in a continuous manner over a given period of time. In some examples, the period of time over which the sensor values are acquired may be the duration of a vehicle trip (e.g., as long as the vehicle is in operation). Acquisition of sensor values may directly correspond to determined human states. For example, heart rate may be monitored by a sensor, and outputted values may correspond to different human states such as drowsy, alert, stressed, and the like.

At 1106, method 1100 includes determining whether a value is greater than the threshold. As noted, if a value is greater than a threshold, the value may be considered critical. Critical values may be values that correspond to human states that affect (e.g., lower) the user's driving performance. Human states that affect the user's driving performance may be considered relevant human states. If the value is greater than the threshold (e.g., is a critical sensor value), method 1100 proceeds to 1108. If the value is not greater than the threshold, the human states being detected may not be considered relevant (e.g., are at baseline), method 1100 returns to 1104 to continue acquiring sensor values. Thus, when the value is not greater than the threshold, no change in vehicle operating state is made and no notification is presented to the driver.

At 1108, method 1100 optionally includes outputting a notification to the user. In some examples, when a relevant value is detected, a human state such as stressed, distracted, drowsy or the like may determined. The notification may be outputted to the user to indicate that the human state is detected and may suggest to the user an action. For example, the notification may indicate to the user that a high anxiety state is detected and suggests that the user should pull over. As another example, the notification may indicate to the user that a distracted state (e.g., based on eye gaze) is detected, either visually or audibly, thereby suggesting that the user pay attention to driving.

At 1110, method 1100 optionally includes adjusting vehicle operating state(s). In some examples, the vehicle may be equipped with an ADAS, such as adaptive cruise control and lane adjustment. In some examples, sensor values greater than the threshold (e.g., critical values) may trigger the ADAS to adjust one or more vehicle operating states. For example, a sensor value above the threshold may trigger the system to activate adaptive cruise control, for example when the human state that is detected is a distracted state. As another example, a sensor value above the threshold may trigger the system to decrease vehicle speed and pull over, for example when the human state that is detected is a drowsy state. Alternatively, a vehicle speed may simply be reduced in response to detection of a critical value.

As described with respect to FIG. 1, the threshold that is determined may be personalized to the particular driver. In this way, the human states that are detected, especially those detected when the value is above the threshold, may be tuned to the specific driver. As individual drivers may have different responses to the same scenario, their human states may be different for the same values. Thus, determining a personalized threshold value may allow for more accurate determination of human states and thus more precise execution of notifications or operating state adjustments.

Further, the threshold value may be continuously updated. For example, acquired sensor values may be fed back into method 100 as historical record data over time. In this way, the threshold value may continue to be updated as the driver's habits, behaviors, and human states evolve over time.

As a non-limiting example, a scenario for a vehicle may include various factors that may affect human state of a driver, such as driving at high speed (e.g., greater than 60 miles per hour, for example), talking on the phone while driving, and another vehicle approaching from a side (e.g., merging into a lane occupied by the vehicle or merging into an adjacent lane). These factors may affect different drivers' human states in different ways. Based on historical record data, according to the method 100 described above, a first driver may have a first threshold value defined therefore for a sensor configured to detect heart rate, a second driver may have a second threshold value defined for the sensor, and a third driver may have a third threshold value defined for the sensor. For example, the first driver may be an inexperienced driver, the second driver may be an average adult driver with typical experience, and the third driver may be an experienced driver (e.g., has racecar driving experience, is a professional drag race, or the like). Thus, the third driver may demand less intervention to vehicle operating states compared to the first and second drivers. Alternatively or additionally, the first, second, and third drivers may have different baseline levels of detectable stress (e.g., different baseline heart rates), which may be accounted for in the historical record data and the defined threshold values therefor.

As an example, in the scenario, a driver (e.g., one of the first, second, and third drivers) may be experiencing a first human state affecting factor, such as driving at high speed. The sensor may output values that are lower than the first, second, and third thresholds when the driver is the first, second, and third drivers, respectively. Then, the driver may begin to experience a second human state affecting factor at the same time, such as talking on the phone. When the driver is the first driver and the first driver is both driving at high speed and talking on the phone, the sensor output value may be greater than the first threshold. As an example, the sensor may detect heart rate and the first and second human state affecting factors may result in an increased heart rate that gives rise to a sensor output value greater than the first threshold value. When the sensor output value for the first driver is greater than the first threshold, one or more operating states of the vehicle may be automatically adjusted in response. For example, adaptive cruise control may reduce a maximum speed by a predefined amount. Further, a lane assist ability may be turned on so as to reduce possibility of the first driver departing from their lane when under the increased stress of the first and second human state affecting factors. Speed reduction when the first driver is affected (e.g., distracted) by the first and second human state affecting factors may be more quickly accomplished as a result of the decreased processing demands put on the vehicle computing system as less sensor output values are demanded to be processed. Thus, speed reduction may be more quickly accomplished.

When the driver is the second driver and the second driver is both driving at high speed and talking on the phone (e.g., experiencing both the first and second human state affecting factors), the sensor output value may not be greater than the second threshold value. As a result, the sensor output values may not be processed for any adjustments in vehicle operating state (e.g., may be ignored). Similarly, when the driver is the third driver and the third driver is both driving at high speed and talking on the phone, the sensor output value may not be greater than the third threshold and the sensor output value may be ignored by the computing device.

Then, in the scenario, another vehicle may be detected as approaching from a side (e.g., merging into the lane the vehicle occupies or merging into an adjacent lane). The driver's peripheral vision may detect this approach vehicle, thereby causing an increase in the driver's heart rate. When the driver is the second driver, the sensor output value with this increased heart rate may be greater than the second threshold when the second driver experiences the first, second, and third human state affecting factors. When the sensor output value for the first driver is greater than the first threshold, one or more operating states of the vehicle may be automatically adjusted in response. For example, adaptive cruise control may reduce a maximum speed by a predefined amount. Further, a lane assist ability may be turned on so as to reduce possibility of the second driver departing from their lane when under the increased stress of the first, second, and third human state affecting factors. When the other vehicle is approaching from the side, a lane departure of the part of the vehicle may be undesirable. Thus, reduced processing demands put on the vehicle computing system, by reducing how many sensor output values are processed and considered by the ADAS, may result in faster reaction by the vehicle when the critical value is detected for the second driver. In this way, lane assist may be more quickly turned on, reducing the possibility of a lane departure as the other vehicle approaches.

When the driver is the third driver and the third driver is experiencing all three of the first, second, and third human state affecting factors, the sensor output value may still be below the third threshold. For example, the third driver, due to their experience, may be less affected by the human state affecting factors and thus the sensor output value may not rise above the third threshold. Again, when the sensor output value is not greater than the third threshold (e.g., is not a critical value), the sensor output value may be ignored and not processed. When not processed, no adjustments to vehicle operating state may be made. Thus overall processing demands of the computing device may be reduced, allowing for faster implementation of adjustments when a critical value is indeed detected.

In this way, personalization of human state detection via individualized threshold values for sensors, may allow for more accurate detection of human states and therefore more appropriate adjustment of vehicle operating states. Further, by personalizing threshold values, the amount of sensor output values that are processed and/or taken into account for adjustment of vehicle operation may be reduced, increasing the efficiency of processing of the computing device of the vehicle and increasing reaction times of the vehicle to implement adjustments via the increased processing efficiency. Further, detection of a critical value may more reliably indicate a relevant human state of the driver.

FIG. 3 shows a data-processing device 300, for example a computer or other computing device. The data-processing device 300 may be incorporated into a vehicle computing system, in some examples. The data-processing device 300 comprises a processor 310 and one or more human state sensors 320 being communicatively coupled to the processor 310. The processor 310, the sensors 320 or the data-processing device 300 may be arranged or comprised by a vehicle. The processor 310 is configured to perform one or more of the method steps of methods 100 and 200 described with reference to FIGS. 1 and 2, using output values of the sensor 320.

The one or more sensors 320 may be configured for acquiring information about a user (e.g., a driver) of the vehicle. For example, the one or more sensors 320 may comprise infrared (IR) sensors configured for tracking eyegaze and eyelid information, heart rate, and the like. The one or more sensors 320 may also comprise vehicle environment telemetry sensors that indicate metrics such as position within a lane, steering wheel position, pedal position, speed, acceleration, yaw rate, and the like. In other examples, the one or more sensors 320 may additionally comprise systems for detection, prediction, and evaluation of human state or performance.

The data-processing device 300 further comprises a memory 330 that is communicatively coupled with at least one of the processor 310 and the sensor(s) 320. The memory 330 is a, in particular non-transitory, computer-readable storage medium. The memory 330 comprises, i.e. stores instructions which, when executed by the data-processing device 300, in particular by the processor 310, cause the data-processing device 300 to carry out one or more of the method steps of methods 100 and 200 described with reference to FIGS. 1 and 2. In other words, the memory 330 comprises, i.e. stores a computer program, the computer program comprising instructions which, when the program is executed by the data-processing device 300, in particular by the processor 310, cause the data-processing device 300 to carry out one or more of the method steps of methods 100 and 200 described with reference to FIGS. 1 and 2.

In some examples, the data-processing device 300, and thus the one or more sensors 320, as herein described may be in communication with an ADAS of the vehicle. For example, sensor output values that are determined and processed may be sent to the ADAS when critical. For example, a critical value may be sent to the ADAS, which may ingest the critical value and adjust one or more operating states of the vehicle based thereon. Further, non-critical values may not be sent to the ADAS.

### REFERENCE SIGNS

100 Method for calibrating a mental and/or physiological state sensor
100-160 Method steps of method 100
200 Method for improving validity of the calibration method 100
210-230 Method steps of method 100
300 Data processing device
310 Processor
320 Sensor
330 Memory
400 Graph
402 Plot
500 Frequency of detection histogram
600 Frequency histogram
700 Integral value histogram
800 Histogram
850 Histogram

1. A vehicle system, comprising:
a vehicle including a sensor and an advanced driver assistance system (ADAS), the sensor being configured to detect one or more parameters of a driver, the sensor mounted in the vehicle and communicating with the ADAS system; and
a computing device comprising a processor and non-transitory memory storing instructions executable by the processor that, when executed, cause the processor to:
obtain sensor output values;
determine, based on the sensor output values, a frequency of detection of each of the sensor output values;
determine based the frequency of detection of each of the sensor values, an integral distribution function of the sensor output values;
determine, based on the integral distribution function, a threshold sensor value; and
in response to detecting a sensor value greater than the threshold sensor value, outputting a notification to a user and/or adjusting one or more vehicle operating states.

2. The vehicle system of claim 1, wherein the sensor value greater than the threshold sensor value is a critical sensor value indicative of a relevant human state.

3. The vehicle system of claim 1, wherein the frequency of detection is determined for sub-ranges of values.

4. The vehicle system of claim 1, wherein, to determine the frequency of detection, the computing device is further configured with instructions stored in non-transitory memory that when executed cause the processor to generate a histogram including frequencies of the sensor output values.

5. The vehicle system of claim 4, wherein, to determine the integral distribution function, the computing device is further configured with instructions stored in non-transitory memory that when executed cause the processor to generate an integral value histogram indicating percent values greater than a left bound for each bin of the integral value histogram.

6. The vehicle system of claim 15, wherein the threshold value is a value of the left bound of a given bin of the integral value histogram for which the percentage value greater than the left bound is 5% or less.

7. The vehicle system of claim 1, wherein, to adjust one or more vehicle operating states in response to detection of the sensor value greater than the threshold value, the computing device is configured with instructions in non-transitory memory that when executed cause the computing device to adjust one or more of a vehicle speed and an advanced driver assistance system parameter.

8. The vehicle system of claim 1, wherein the notification outputted in response to detection of the sensor value greater than the threshold value, is presented to the driver one or more of visually and audibly.

9. The vehicle system of claim 1, wherein, in response to detection of a second sensor value lower than the threshold value, the computing device is configured with instructions in non-transitory memory that when executed cause the processor to continue acquiring sensor values without outputting a notification or adjusting a vehicle operating state.

10. A vehicle system, comprising:
a vehicle including a sensor and an advanced driver assistance system (ADAS), wherein the sensor is configured to detect one or more parameters of a driver, the sensor mounted in the vehicle and communicating with the ADAS; and
a computing device comprising a processor and non-transitory memory storing instructions executable by the processor that, when executed, cause the processor to:
obtain sensor output values;
determine, based on the sensor output values, a frequency of detection of each of the sensor output values;
determine, based on the frequency of detection of each of the sensor values, an integral distribution function of the sensor output values;
determine, based on the integral distribution function, a threshold sensor value, wherein a sensor value greater than the threshold sensor value is a critical sensor value indicative of a relevant human state;
obtain sensor values with the sensor; and
in response to detecting a critical sensor value among the obtained sensor values, outputting a notification to the driver and/or adjusting one or more vehicle operating states, wherein normal and uncritical fluctuations of the sensor values are ignored such that only outliers of the sensor values are determined to be critical so that both driver and sensor behaviour are considered when interpreting sensor values.

11. The vehicle system of claim 10, wherein, when the threshold sensor value is a first threshold value, the critical sensor value is detected when the driver is experiencing a first human state affecting factor.

12. The vehicle system of claim 11, wherein, when the threshold sensor value is a second threshold value, the obtained sensor values do not comprise a value greater than the second threshold value when the driver is experiencing the first human state affecting factor.

13. The vehicle system of claim 12, wherein when the threshold sensor value is the second threshold value, the critical sensor value is detected when the driver is experiencing both a first and second human state affecting factor.

14. The vehicle system of claim 10, wherein determining the threshold sensor value based on the integral distribution function comprises determining a boundary value that 5% or less of the sensor output values are greater than.

15. The vehicle system of claim 14, wherein the frequency of detection of each of the sensor values is represented by a frequency histogram and the integral distribution function is represented by an integral value histogram, wherein the boundary value is a left bound of a bin of the integral value histogram.
